SEQUENCE LISTING (4 sequences)

SEQ ID NO: 1 - DNA, 28 bp, Artificial
  Primer
ataaggatccatgactaaaagcaacgga

SEQ ID NO: 2 - DNA, 28 bp, Artificial
  Primer
tattgatatcctacatcttggtttcact

SEQ ID NO: 3 - DNA, 28 bp, Artificial
  Primer
ataaggatccatggggaaaccggcgagg

SEQ ID NO: 4 - DNA, 28 bp, Artificial
  Primer
tattgatatcctagaactgtgaggtctg

(19) United States
(12) Patent Application Publication
(10) Pub. No.: US 2007/0142467 A1
(43) Pub. Date: Jun. 21, 2007

(54) 3-alkylaryl aspartate compounds and their use for selective enhancement of synaptic transmission

(75) Inventors: Christopher S. Esslinger, Alberton, MT (US); Richard J. Bridges, Clinton, MT (US); Michael P. Kavanaugh, Missoula, MT (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: The University of Montana, Missoula, MT (US)

(21) Appl. No.: 11/256,583

(22) Filed: Oct. 21, 2005

Related U.S. Application Data
(60) Provisional application No. 60/620,946, filed on Oct. 21, 2004.

(51) Int. Cl. A61K 31/277; A61K 31/198; C07C 255/28; C07C 229/38

(52) U.S. Cl. 514/520; 514/566; 558/406; 562/443

(57) ABSTRACT

The present invention provides an L-aspartate derivative compound represented by the following structure 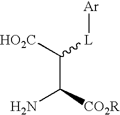
wherein Ar represents an aromatic group; L represents a linking moiety; R represents hydrogen, alkyl, aryl, or heteroaryl; and [symbol] indicates that the stereochemistry at the 3-position can be R or S. The compounds of the invention are useful for selectively inhibiting EAAT3 and for enhancing synaptic transmission. Additionally, the inventive compounds can be used to treat a patient suffering from Alzheimers disease or a neuropathy or a neurodegenerative disease in which L-glutamate transporter activity is involved in the onset of the disease.

### CROSS-REFERENCE TO RELATED APPLICATIONS

This patent application claims the benefit of U.S. Provisional Patent Application No. 60/620,946, filed Oct. 21, 2004.

### STATEMENT REGARDING FEDERALLY SPONSORED RESEARCH AND DEVELOPMENT

This invention was made in part with Government support under Grant Numbers R01 NS32270 and P20 RR15583 awarded by the National Institutes of Health. The Government may have certain rights in this invention.

### BACKGROUND OF THE INVENTION

The high-affinity, sodium-dependent excitatory amino acid transporters (EAATs) are involved in regulating extracellular L-glutamate levels in the mammalian central nervous system (Danbolt, N. C. Prog. Neurobiol. 2001, 65 (1), 1-105. Maragakis, N. J.; Rothstein, J. D. Neurobiol. Dis. 2004, 15, 461-473. Bridges, R. J.; Esslinger, C. S. Pharmacol. Ther. 2005, 107 (3), 271-285.). L-glutamate is the primary excitatory neurotransmitter in these systems and participates in standard fast synaptic communication, as well as in higher order types of signal processing linked to development, synaptic plasticity, learning, and memory (Balazs et al., 2005). Thus, glutamate-mediated neuronal damage is reported to be a contributing pathological mechanism in both acute CNS injury (e.g., stroke, head trauma, spinal cord injury) and chronic neurodegerneative disease (e.g., amyotrophic lateral sclerosis, Alzheimer's disease, Huntington's disease) (Choi, D. W. Prog Brain Res. 1994, 100, 47-51. Mattson, M. P. Neuromol. Med. 2003, 3 (2), 65-94. Olney, J. W. Curr, Opin. Pharmacol. 2003, 3 (1), 101-109.). The ability of EAATs to sequester L-glutamate into neurons and glia in an efficient manner places these membrane proteins in a position where they can influence the amount and/or time course with which glutamate is in contact with EAA receptors within the context of either normal signaling or pathologic injury.

The five subtypes types of EAATs (EAAT1-EAAT5) share a level of homology of about 50-60% with one another, as well as a dependency on sodium and a high affinity for L-glutamate, but each exhibits a distinct anatomical and cellular distribution (Furuta, A.; Martin, L. J.; Lin, C.-L. G.; Dykes-Hoberg, M.; Rothstein, J. B. Neuroscience 1997, 81 (4), 1031-1042. Gegelashvili, G.; Schousboe, A. Brain Res. Bull. 1998, 45 (3), 233-238. Seal, R. P.; Amara, S. G. Annu. Rev. Pharmacol. Toxicol. 1999, 39, 431-456. Danbolt, N. C. Prog. Neurobiol. 2001, 65 (1), 1-105. Maragakis, N. J.; Rothstein, J. D. Neurobiol. Dis. 2004, 15, 461-473.).

Given the role of EAAT3 in synaptic transmission and plasticity, developing a selective EAAT3 inhibitor would provide an important advance and a tool useful in studying such phenomena and also potentially in modulating synaptic transmission and plasticity. Significant advances have been made in generating inhibitors and substrates that can be used to assess EAAT activity with little or no cross-reactivity with EAA receptors, such as L-trans-2,4-pyrrolidine dicarboxylate (L-trans-2,4-PDC) and β-threo-benzyloxy-aspartate (TBOA) (Bridges, R. J.; Esslinger, C. S. Pharmacol. Ther. 2005, 107 (3), 271-285.). Limiting the positions that can be occupied by required functional groups (e.g., carboxylate and amino groups) has the potential to both increase potency and reduce cross-reactivity. Unfortunately, identifying compounds that readily differentiate among the individual EAAT subtypes has proven to be more elusive than developing analogues that discriminate glutamate transporters from glutamate receptors. The exception to this limitation has been the progress made in pharmacologically delineating EAAT2, particularly within the context of the most commonly studied EAATs 1-3. The glutamate analogues dihydrokainate (DHK), L-anti-endo-3,4-methanopyrrolidine dicarboxylate (L-anti-endo-3,4-MPDC) (Bridges, R. J.; Lovering, F. E.; Koch, H.; Cotman, C. W.; Chamberlin, A. R. Neurosci. Lett. 1994, 174, 193-197.), S-2-amino-3-(3-hydroxy-1,2,5-thiadiazol-5-yl)propionic acid (TDPA) (Brauner-Osborne et al., 2000), and 3-amino-tricyclo[2.2.1.0^{2.6}]heptane-1,3-dicarboxylate (WAY-855) (Dunlop, J.; Eliasof, S.; Stack, G.; McIlvain, H. B.; Greenfield, A.; Kowal, D. M.; Petroski, R.; Carrick, T. Br. J. Pharmacol. 2003, 140, 839-846.) potently and preferentially interact with EAAT2. Thus there remains a use for an agent that selectively blocks EAAT3.

### BRIEF SUMMARY OF THE INVENTION

The present invention provides an L-aspartate derivative compound represented by the following structure (I) 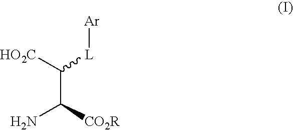
wherein Ar represents an aromatic group; L represents a linking moiety; R represents hydrogen, alkyl, aryl, or heteroaryl; and [symbol] indicates that the stereochemistry at the 3-position can be R or S.

In one aspect, the compounds of the invention can be used for selectively attenuating the activity of EAAT3. Additionally, the inventive compounds can be useful for enhancing synaptic transmission. In another aspect, the inventive compounds can be used to treat a patient suffering from Alzheimers disease or a neuropathy or a neurodegenerative disease in which L-glutamate transporter activity is involved in the onset of the disease. The invention also provides a pharmaceutical composition comprising the inventive compounds and a pharmaceutically acceptable carrier, which can be administered to facilitate treatment of such conditions. These and other advantages of the invention, as well as additional inventive features, will be apparent from the accompanying drawings and from the description of the invention provided herein.

### BRIEF DESCRIPTION OF THE DRAWING(S)

FIG. 1 depicts representative Lineweaver-Burk plots of single experiments demonstrating 2(S),3(S)-3-benzyl aspartic acid as a competitive inhibitor of ³H-D-aspartate uptake by (A) hEAAT1, (B) hEAAT2 and (C) hEAAT3 expressed in C17.2 cells. The inset within each graph shows a replot of K_{Mapp} vs [2(S),3(S)-3-benzyl aspartic acid] that was used to determine the indicated Kᵢ value for the depicted experiment.

FIG. 2(A) shows inhibition of hEAAT3 transport by L-3-benzyl aspartic acid in a representative oocyte voltage-clamped at −30 mV (glutamate and inhibitor applied for the durations indicated by corresponding bars above traces).

FIG. 2(B) shows parallel glutamate dose-response shift with increasing [3-benzylaspartate] is consistent with competitive inhibition. Data points represent mean +/− SEM for 3-5 oocytes.

FIG. 2(C) shows a Schild analysis of L-3-benzyl aspartic acid inhibition in analogous competition experiments with EAATs 1, 2, and 3 (slope values constrained to 1) yielded K_{D} values of 12, 9, and 2 μM, respectively.

FIG. 3 shows that 3-benzyl-L-aspartate affects hippocampal CA1 synaptic transmission in conditions of increased release probability. 3-benzyl-L-aspartate prolongs the time course of the second EPSP elicited by paired pulse stimulation with no effect on the time course of the first EPSP. Left: control; Right: 30 μM 3-benzyl-L-aspartate. Scale bars=1mV, 100 ms.

### DETAILED DESCRIPTION OF THE INVENTION

The invention provides an L-aspartate derivative compound represented by the following structure (I) 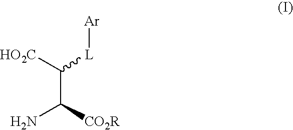
wherein Ar represents an aromatic group; L represents a linking moiety; R represents hydrogen, alkyl, aryl, or heteroaryl; and [symbol] indicates that the stereochemistry at the 3-position can be R or S. The invention also encompasses pharmaceutically acceptable salts, solvates, and hydrates of the inventive compounds.

The linking moiety, L, represents any suitable linking moiety. Preferably, L comprises a covalent bond, straight or branched C₁₋₆alkyl, straight or branched C₂₋₈alkenyl, or straight or branched C₂₋₈alkynyl, each optionally substituted with C₁₋₃alkyl, hydroxyl, amino, nitro, cyano, carboxyl, or halogen. When L is a covalent bond, Ar is attached directly to the 3-position of the aspartate moiety. When L is alkyl, preferably the alkyl chain comprises 1-3 carbon atoms, for example, 1 carbon atom (methylene) or 2 carbon atoms (ethylene). When L is alkenyl, preferably the alkenyl group comprises 2-4 carbon atoms, for example 3 carbon atoms, or —CH₂—CH═CH—, which can be oriented in either direction.

The aromatic group, Ar, represents any suitable aromatic group. Preferably, Ar represents an optionally substituted C₅₋₃₀ aromatic group that can comprise 1-5 fused rings and 0, 1, 2, 3, 4, or 5 heteroatoms selected from O, N, or S. Preferably, Ar is phenyl, naphthyl, anthracenyl, phenanthyl, furyl, thiophenyl, or pyrrolyl. The aromatic group can be further substituted with at least one substituent selected from the group consisting of a C₁₋₆ alkyl group, a C₂₋₆alkenyl group, a C₅₋₁₀aryl group, a C₁₋₆alkoxy group, a hydroxy group, an amino group, a nitro group, a cyano group, a carboxyl group, and a halogen. Preferably Ar is substituted with a nitro group, a C₁₋₆alkyl group, or two methyl groups.

The substituent R is preferably hydrogen, straight or branched C₁₋₆alkyl, straight or branched C₂₋₈alkenyl, or straight or branched C₂₋₈alkynyl, each optionally substituted with C₁₋₃alkyl, hydroxyl, amino, nitro, cyano, carboxyl, halogen, or an optionally substituted C₅₋₃₀ aromatic group that can comprise 1-5 fused rings and 0, 1, 2, 3, 4, or 5 heteroatoms selected from O, N, or S, such as phenyl, naphthyl, anthracenyl, phenanthyl, furyl, thiophenyl, or pyrrolyl; or R is an optionally substituted C₅₋₃₀ aromatic group that can comprise 1-5 fused rings and 0, 1, 2, 3, 4, or 5 heteroatoms selected from O, N, or S, such as phenyl, naphthyl, anthracenyl, phenanthyl, furyl, thiophenyl, or pyrrolyl.

The inventive compounds can exist as enantiomers. The invention includes L-aspartate derivatives with stereochemical configurations at the 3-position of R and S. The enantiomers can exist in the substantially pure form, such as >90% R or >90% S, for example, >95% R or >95% S, or specifically >99% R or >99% S. The invention also includes mixtures of enantiomers, such as R:S from about 1:10 to about 10:1, specifically about 1:1, about 1:2, or about 2:1.

Preferred compounds of the invention include those wherein Ar is phenyl, L is methylene, R is hydrogen, and the stereochemistry at the 3-position is R or S, or the compounds can exist as a 1:2 mixture of R and S enantiomers, wherein the R or S designation represents the stereochemistry at the 3-position of the L-aspartate moiety. In an additional embodiment of the invention, Ar is phenyl, L is ethylene, and R is hydrogen. Another preferred compound is one in which Ar is 3,5-dimethylphenyl, L is methylene, and R is hydrogen. In another preferred embodiment, Ar is naphthyl, L is methylene, and R is hydrogen. Another compound of the invention is one in which Ar is phenyl, L is —CH₂—CH═CH—, and R is hydrogen. A further embodiment of the invention is the compound in which Ar is 4-nitrophenyl, L is methylene, and R is hydrogen. Also preferred is the compound in which Ar is 4-nitronaphthyl, L is methylene, and R is hydrogen. Exemplary compounds of the invention are set forth below in structures (II)-(X).

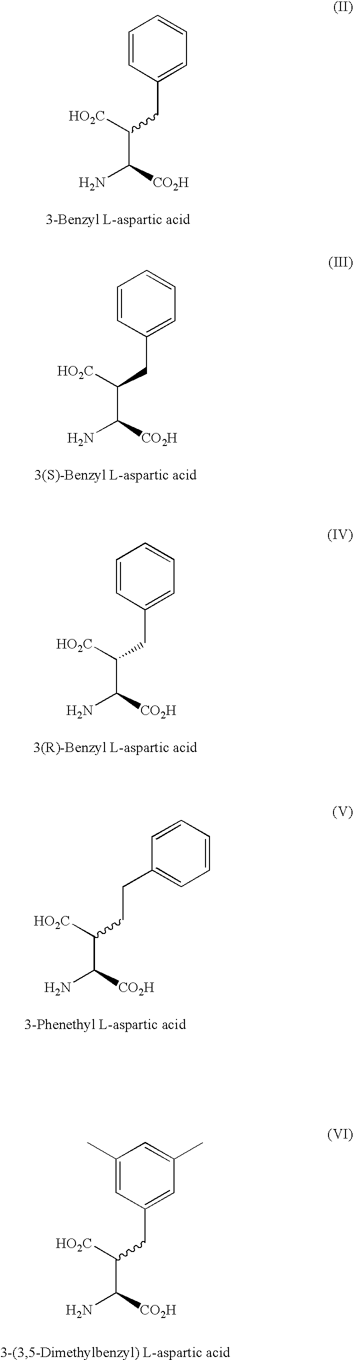

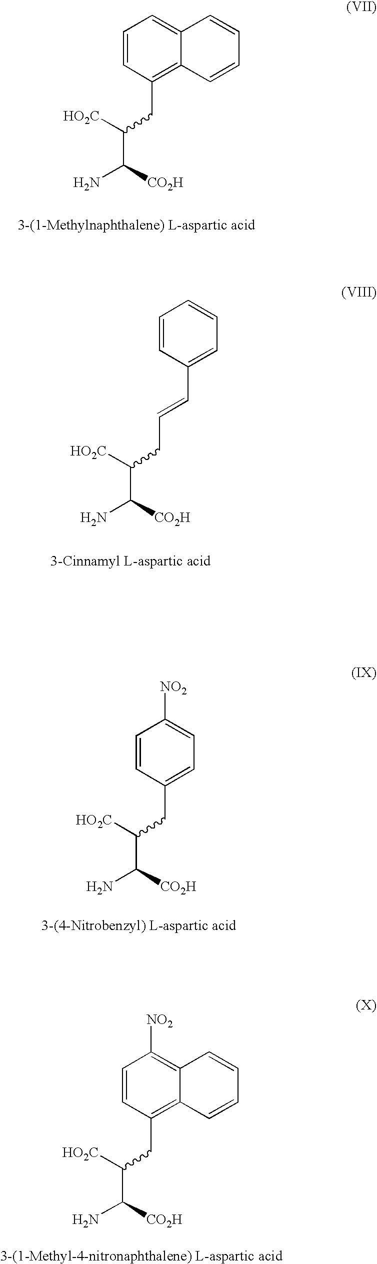

The invention further provides a method of preparing the inventive compounds. In general, the inventive compounds can be prepared from L-aspartic acid. In accordance with the inventive method, neat thionyl chloride is added dropwise to a solution of L-aspartic acid in methanol and stirred at room temp. The reaction mixture is then concentrated in vacuo and chased with methanol and methylene chloride using the rotovapor to yield L-aspartate dimethyl ester hydrochloride.

The dimethyl aspartate hydrochloride is then suspended in methylene chloride (dried with magnesium sulfate) followed by the addition of trityl chloride, with subsequent dropwise addition of triethylamine. The mixture is stirred at room temperature, after which the reaction mixture is diluted with ether and filtered through a plug of silica gel followed by a mixture of about 30% ethyl acetate and about 70% hexanes to wash the silica. The filtrates are combined and concentrated to yield N-trityl L-aspartate dimethyl ester.

The N-trityl L-aspartate dimethyl ester dissolved in THF is chilled to about −30° C. under argon followed by the addition of approx. 2M lithium hexamethyldisilazide/THF solution and stirred at about −30° C., after which X—L—Ar (X is a halogen, preferably Br or I) is added dropwise in THF. The mixture is warmed to about −5° C. and stirred for about 1 hr. under argon. The reaction is then quenched with approx. 1M solution of ammonium chloride, and diluted with diethyl ether. The mixture is allowed to warm to room temperature, is separated, and the organic layer is washed with brine and dried with sodium sulfate. The drying agent is then filtered off using a silica plug with a mixture of about 30% ethyl acetate and about 70% hexanes used to wash the silica, and the organic solution concentrated to yield the crude product, which is used in the next step without further purification.

After the foregoing, approx. 6N HCI is added to the crude N-trityl-β-substituted L-aspartate dimethyl ester and the mixture is refluxed. The mixture is then concentrated, taken up in a minimum amount of water, washed with ethyl acetate, and concentrated. The residue is then taken up in a minimum amount of water, neutralized with sodium hydroxide, and loaded onto a column containing ion exchange resin (preferably Ag-1 2×acetate form). The column is washed with about 8 column volumes of water followed by elution of acetic acid solutions of increasing molarity. The desired mixture of 2(S),3(S)- and 2(S),3(R)-3-substituted aspartates are combined, concentrated, and chased with water to yield the inventive L-aspartate derivatives as a mixture of diastereomers.

The diastereomeric mixture can be separated by conventional methods known to those of skill in the art to be useful for such separations, such as HPLC.

In another aspect, the invention provides a method of selectively attenuating the activity of EAAT3 in a cell. In accordance with this method, compounds of the invention are administered to a cell in an amount sufficient to attenuate the activity of EAAT3 in the cell. Exemplary compounds for use in the method are described herein as formulae I, II, III, IV, V, VI, VII, VIII, IX, and X. Preferably, the compound selectively inhibits EAAT3. In this context, “selective” inhibition is assessed using a Kᵢ value (or similar measure of inhibition) for EAAT3 compared to that for other EAATs.

The attenuation of EAAT3 can be measured by any method known to those of skill in the art. One such method is measuring relative levels of functional D-[³H]-aspartate uptake as described in Example 8. Alternatively, attenuation of EAAT1-3 activity can be determined by measuring transporter-mediated current in Xenopus oocytes, as set forth in Example 9.

The method of the invention will preferably reduce the activity of EAAT3 in the cell by at least about 25%, more preferably by at least about 50%, such as by at least about 75%, for example by at least about 90%. Even more preferably, the method will reduce the activity of EAAT3 in the cell by at least about 95%, such as by at least about 97%, or at least about 99%. In preferred embodiments, the method will substantially inhibit or even almost completely inhibit the activity of EAAT3 in the cell.

In another aspect, the invention provides a method of enhancing synaptic transmission. This is accomplished by administration of the inventive compound to a neural synapse in an amount sufficient to enhance synaptic transmission at the synapse. Exemplary compounds for use in the method are described herein as formulae I, II, III, IV, V, VI, VII, VIII, IX, and X. Assessment of the enhancement of synaptic transmission can be measured using any suitable method known to those of skill in the art, such as electrophysiological recording of synaptic transmission as described in Example 10.

The method of the invention will preferably enhance synaptic transmission by at least about 5%, more preferably by at least about 10%, such as by at least about 25%, for example by at least about 50%. Even more preferably, the method will enhance synaptic transmission by at least about 75%, such as by at least about 90%, or at least about 100%.

The invention further provides the use of the inventive compounds in medicine. The inventive compounds can be used for the preparation of a medicament suitable for treating a neuropathy or a neurodegenerative disease, such as, wherein L-glutamate transporter activity is involved in the onset of the disease. As such, the invention provides a method for treating a patient suffering from a neuropathy or a neurodegenerative disease, for example, wherein L-glutamate transporter activity is involved in the onset of the disease. In accordance with this method, one or more inventive compounds are administered to the patient in an amount sufficient to treat the neuropathy or neurodegenerative disease or symptoms thereof. Exemplary compounds for use in the method are described herein as formulae I, II, III, IV, V, VI, VII, VIII, IX, and X. Treating a neuropathy or neurodegenerative disease can be achieved successfully by reducing or alleviating some or all of the symptoms of the disease, as can be assessed by certain diagnostic methods known to those of skill in the art. In some cases, it is desirable for the method to slow or even halt or reverse progression of the neuropathy or neurodegenerative disease.

The invention further provides the use of the inventive compounds for the preparation of a medicament suitable for treating Alzheimer's disease and a method of treatment of Alzheimer's disease in a patient. In accordance with this method, one or more inventive compounds are administered to the patient in an amount sufficient to treat Alzheimer's disease or symptoms thereof. Exemplary compounds for use in the method are described herein as formulae I, II, III, IV, V, VI, VII, VIII, IX, and X. Treating Alzheimer's disease or the symptoms thereof is herein defined as reducing or alleviating some or all of the symptoms of the disease, as can be assessed by certain diagnostic methods known to those of skill in the art. In some cases, it is desirable for the method to slow or even halt or reverse progression of Alzheimer's disease.

The dose administered to an animal, particularly a human, in the context of the present invention should be sufficient to effect a therapeutic response in the animal over a reasonable time frame. The dose will be determined by the strength of the particular compositions employed and the condition of the animal (e.g., human), as well as the body weight of the animal (e.g., human) to be treated. The size of the dose also will be determined by the existence, nature, and extent of any adverse side effects that might accompany the administration of a particular composition.

To facilitate the use of the inventive compounds, the invention provides pharmaceutical compositions comprising an inventive L-aspartate derivative. Preferably, the pharmaceutical compositions further comprise a pharmaceutically acceptable carrier.

One skilled in the art will appreciate that suitable methods of administering an L-aspartate derivative composition of the present invention to an animal, e.g., a mammal such as a human, are known, and, although more than one route can be used to administer a particular composition, a particular route can provide a more immediate and more effective reaction than another route. Pharmaceutically acceptable carriers are also well known to those who are skilled in the art. The choice of carrier will be determined, in part, both by the particular composition and by the particular method used to administer the composition. Accordingly, there is a wide variety of suitable formulations of the pharmaceutical compositions of the present invention.

Formulations suitable for oral administration can consist of (a) liquid solutions, such as an effective amount of the L-aspartate derivative dissolved in diluents, such as water or saline, (b) capsules, sachets or tablets, and the like, each containing a predetermined amount of the active ingredient, as solids or granules, (c) suspensions in an appropriate liquid, and (d) suitable emulsions.

Tablet forms can include one or more of lactose, mannitol, cornstarch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, such carriers as are known in the art.

The L-aspartate derivatives of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like.

Formulations suitable for parenteral administration include aqueous and non-aqueous solutions, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The following examples further illustrate the invention but, of course, should not be construed as in any way limiting its scope.

### EXAMPLE 1

This example demonstrates the synthesis of 3-benzyl L-aspartic acid (Compound II).

L-Aspartate dimethyl ester hydrochloride: To L-aspartic acid (13.5 g, 100 mmol) in 100 ml methanol was added dropwise neat thionyl chloride and stirred at room temp for 48 hrs. The reaction was then concentrated in vacuo and chased with methanol (3×30 ml) and methylene chloride (3×30 ml) using the rotovapor to yield L-aspartate dimethyl ester hydrochloride (19.5 g, quant. yield) as a white powder.

N-trityl L-aspartate dimethyl ester: The dimethyl aspartate hydrochloride (9.85 g, 50 mmol) was suspended in 50 ml methylene chloride (dried with magnesium sulfate) followed by the addition of trityl chloride (132.4 g, 47.5 mmol, 0.95 eq), with subsequent dropwise addition of triethylamine (15.2 g, 21 ml, 150 mmol, 3.0 eq). The mixture was stirred at room temperature for 3 hr, after which the reaction mixture was diluted with 100 ml ether and filtered through a plug of silica gel followed by a 30% ethyl acetate 70% hexanes mixture to wash the silica. The filtrates were combined and concentrated to yield N-trityl L-aspartate dimethyl ester (19.1 g, 95%) as a light yellow crystalline solid.

N-trityl-3-benzyl L-aspartate dimethyl ester: The N-trityl L-aspartate dimethyl ester (40.3 g, 100 mmol, 1.0 eq.) dissolved in 100 ml THF (SureSeal bottle) was chilled to −30° C. under argon followed by the addition of 110 ml of 2M lithium hexamethyldisilazide/THF solution (220 mmol, 2.2 eq.) and stirred at −30° C. for approximately 30 min., at which time benzyl bromide (25.6 g, 150 mmol, 1.5 eq.) was added dropwise in 50 ml THF. The mixture was warmed to −5° C. and stirred for 1 hr. under argon. The reaction was then quenched with a 1M solution of ammonium chloride (200 mmol, 2 eq.), and diluted with 100 ml diethyl ether. The mixture was allowed to warm to room temperature, separated, and the organic layer washed with brine and dried with sodium sulfate. The drying agent was then filtered off using a silica plug with a 30% ethyl acetate 70% hexanes mixture used to wash the silica, and the organic solution concentrated to yield the crude product N-trityl-3-benzyl L-aspartate dimethyl ester (57 g) as a tan oil containing benzyl bromide as a contaminant. This oil was used in the next step without further purification.

3-Benzyl L-aspartic acid (II): To the crude N-trityl-3-benzyl L-aspartate dimethyl ester (20 g, ca. 15 g actual, 31 mmol) was added 30 ml and 30 ml 6N HCI and the mixture refluxed for 6 hr. The mixture was concentrated, taken up in a minimum amount of water, washed with ethyl acetate, and concentrated to yield 10.3 g brown solid. The residue was then taken up in a minimum amount of water, neutralized with sodium hydroxide, and loaded onto a column containing ion exchange resin (Ag-1 2×acetate form, 150 g). The column was washed with 8 column volumes of water followed by elution of acetic acid solutions of increasing molarity (0.1M×500 ml, 0.2M×500 ml, 0.5M×500 ml, 1M×1000 ml, 2M×2000 ml, 5M×500 ml). the desired mixture of 2(S),3(S)- and 2(S),3(R)-3-benzyl aspartates (2M and 5M fractions) were combined, concentrated, and chased with water (3×50 ml) to yield 3-benzyl L-aspartic acid (5.75 g, 83%) as a mixture of diastereomers.

¹H-NMR suggest the aryl derivatives are approximately 1:1 to 2:1 mixtures of diastereomers [2(S)3(S):2(S)3(R)]. The diastereomeric mixture can be separated by HPLC using reverse phase C18 column and 0.1M NH₄OAc pH=6.4 as the mobile phase (elution of the SS isomer (III) first, followed by the SR (IV) isomer). EXAMPLE 2

This example demonstrates the synthesis of 3-(4-nitrobenzyl) L-aspartic acid (Compound IX).

N-trityl-3-(4-nitrobenzyl) L-aspartate dimethyl ester: The N-trityl L-aspartate dimethyl ester (4.03 g, 10 mmol, 1.0 eq.) dissolved in 10 ml THF (SureSeal bottle) was chilled to −30° C. under argon followed by the addition of 11 ml of 2M lithium hexamethyldisilazide/THF solution (22 mmol, 2.2 eq.) and stirred at −30° C. for approximately 30 min. at which time p-nitrobenzyl bromide (3.24 g, 1.5 mmol, 1.5 eq.) was added dropwise in 5 ml THF. The mixture was warmed to −5° C. and stirred for 30 min. under argon. The reaction was then quenched with a 1M solution of ammonium chloride (20 mmol, 2 eq.), and diluted with 50 ml diethyl ether. The mixture was allowed to warm to room temperature, separated, and the organic layer washed with brine and dried with sodium sulfate. The drying agent was then filtered off using a silica plug with a 30% ethyl acetate 70% hexanes mixture used to wash the silica, and the organic solution concentrated to yield the crude product N-trityl-3-(4-nitrobenzyl) L-aspartate dimethyl ester (5.7 g) as a tan oil containing 4-nitrobenzyl bromide as a contaminant. This oil was used in the next step without further purification.

3-(4-nitrobenzyl) L-aspartic acid (IX): To the crude N-trityl-3-(4-nitrobenzyl) L-aspartate dimethyl ester (4.0 g, , 7.4 mmol) was added 5 ml and 5 ml 6N HCI and the mixture refluxed for 6 hr. The mixture was concentrated, taken up in a minimum amount of water, washed with ethyl acetate, and concentrated to yield 3.2 g brown solid. The residue was then taken up in a minimum amount of water, neutralized with sodium hydroxide, and loaded onto a column containing ion exchange resin (Ag-1 2×acetate form, 45 g). The column was washed with 8 column volumes of water followed by elution of acetic acid solutions of increasing molarity (0.1M×50 ml, 0.2M×50 ml, 0.5M×50 ml, 1M×100 ml, 2M×300 ml, 5M×100 ml). The desired mixture of 2(S),3(S)- and 2(S),3(R)-3-(4-nitrobenzyl) aspartates (2M and 5M fractions) were combined, concentrated, and chased with water (3×50 ml) to yield 3-(4-nitrobenzyl) L-aspartic acid (1.1 g, 55%) as a mixture of diastereomers.

### EXAMPLE 3

This example demonstrates the synthesis of 3-(1-methylnaphthalene) L-aspartic acid (Compound VII).

N-trityl-3-(1-methylnaphthalene) L-aspartate dimethyl ester: The N-trityl L-aspartate dimethyl ester (8.006 g, 20 mmol, 1.0 eq.) dissolved in 20 ml THF (SureSeal bottle) was chilled to −30° C. under argon followed by the addition of 22 ml of 2M lithium hexamethyldisilazide/THF solution (44 mmol, 2.2 eq.) and stirred at −30° C. for approximately 30 min., at which time 1-bromomethyl naphthalene (14.6 g, 66 mmol, 1.5 eq.) was added dropwise in 20 ml THF. The mixture was warmed to −5° C. and stirred for 1 hr. under argon. The reaction was then quenched with a 1M solution of ammonium chloride (40 mmol, 2 eq.), and diluted with 100 ml diethyl ether. The mixture was allowed to warm to room temperature, separated, and the organic layer washed with brine and dried with sodium sulfate. The drying agent was then filtered off using a silica plug with a 30% ethyl acetate 70% hexanes mixture used to wash the silica, and the organic solution concentrated to yield the crude product N-trityl-3-(1-methylnaphthalene) L-aspartate dimethyl ester (11.3 g) as a tan oil containing 1-bromomethyl naphthalene as a contaminant. This oil was used in the next step without further purification.

3-(1-methylnaphthalene) L-aspartic acid (VII): To the crude N-trityl-3-(1-methyl naphthalene) L-aspartate dimethyl ester (5 g, 9.2 mmol) was added 10 ml and 10 ml 6N HCI and the mixture refluxed for 6 hr. The mixture was concentrated, taken up in a minimum amount of water, washed with ethyl acetate, and concentrated to yield 4.3 g brown solid. The residue was then taken up in a minimum amount of water, neutralized with sodium hydroxide, and loaded onto a column containing ion exchange resin (Ag-1 2×acetate form, 50 g). The column was washed with 8 column volumes of water followed by elution of acetic acid solutions of increasing molarity (0.1M×100 ml, 0.2M×100 ml, 0.5M×100 ml, 1M×100 ml, 2M×500 ml, 5M×200 ml). The desired mixture of 2(S),3(S)- and 2(S),3(R)-3-(1-methyl naphthalene) aspartates (2M and 5M fractions) were combined, concentrated, and chased with water (3×50 ml) to yield 3-(1-methylnaphthalene) L-aspartic acid (0.42 g, 17%) as a mixture of diastereomers.

### EXAMPLE 4

This example demonstrates the synthesis of 3-(3,5-dimethylbenzyl) L-aspartic acid (Compound VI).

N-trityl-3-(3,5-dimethylbenzyl) L-aspartate dimethyl ester: The N-trityl L-aspartate dimethyl ester (4.03 g, 100 mmol, 1.0 eq.) dissolved in 10 ml THF (SureSeal bottle) was chilled to −30° C. under argon followed by the addition of 11 ml of 2M lithium hexamethyldisilazide/THF solution (22 mmol, 2.2 eq.) and stirred at −30° C. for approximately 30 min. at which time 3,5-dimethyl benzyl bromide (4.0 g, 20 mmol, 2.0 eq.) was added dropwise in 5 ml THF. The mixture was warmed to −5° C. and stirred for 1 hr. under argon. The reaction was then quenched with a 1M solution of ammonium chloride (20 mmol, 2 eq.), and diluted with 50 ml diethyl ether. The mixture was allowed to warm to room temperature, separated, and the organic layer washed with brine and dried with sodium sulfate. The drying agent was then filtered off using a silica plug with a 30% ethyl acetate 70% hexanes mixture used to wash the silica, and the organic solution concentrated to yield the crude product N-trityl-3-(3,5-dimethylbenzyl) L-aspartate dimethyl ester (5.7 g) as a tan oil containing 3,5-dimethylbenzyl bromide as a contaminant. This oil was used in the next step without further purification.

3-(3,5-dimethylbenzyl) L-aspartic acid (VI): To the crude N-trityl-3-(3,5-dimethylbenzyl) L-aspartate dimethyl ester (2.6 g, 5.0 mmol) was added 5 ml and 5 ml 6N HCI and the mixture refluxed for 6 hr. The mixture was concentrated, taken up in a minimum amount of water, washed with ethyl acetate, and concentrated to yield 1.52 g brown solid. The residue was then taken up in a minimum amount of water, neutralized with sodium hydroxide, and loaded onto a column containing ion exchange resin (Ag-1 2×acetate form, 23 g). The column was washed with 8 column volumes of water followed by elution of acetic acid solutions of increasing molarity (0.1M×50 ml, 0.2M×50 ml, 0.5M×50 ml, 1M×100 ml, 2M×200 ml, 5M×100 ml). The desired mixture of 2(S),3(S)- and 2(S),3(R)-3-(3,5-dimethylbenzyl) aspartates (2M and 5M fractions) were combined, concentrated, and chased with water (3×50 ml) to yield 3-(3,5-dimethylbenzyl) L-aspartic acid (0.88 g, 70%) as a mixture of diastereomers.

### EXAMPLE 5

This example demonstrates the synthesis of 3-phenethyl L-aspartic acid (Compound V).

N-trityl-3-phenethyl L-aspartate dimethyl ester: The N-trityl L-aspartate dimethyl ester (1.0 eq.) dissolved in THF (SureSeal bottle) is chilled to −30° C. under argon followed by the addition of 2M lithium hexamethyldisilazide/THF solution (2.2 eq.) and is stirred at −30° C. for approximately 30 min. at which time phenethyl bromide (2.0 eq.) is added dropwise in THF. The mixture is warmed to −5° C. and stirred for 1 hr. under argon. The reaction is then quenched with a 1M solution of ammonium chloride (2 eq.), and diluted with diethyl ether. The mixture is allowed to warm to room temperature, separated, and the organic layer washed with brine and dried with sodium sulfate. The drying agent is then filtered off using a silica plug with a 30% ethyl acetate 70% hexanes mixture used to wash the silica, and the organic solution concentrated to yield the crude product N-trityl-3-phenethyl L-aspartate dimethyl ester containing phenethyl bromide as a contaminant. This is used in the next step without further purification.

3-phenethyl L-aspartic acid (V): To the crude N-trityl-3-phenethyl L-aspartate dimethyl ester is added 6N HCI and the mixture is refluxed for 6 hr. The mixture is concentrated, taken up in a minimum amount of water, washed with ethyl acetate, and concentrated to remove the solvent. The residue is then taken up in a minimum amount of water, neutralized with sodium hydroxide, and loaded onto a column containing ion exchange resin (Ag-1 2×acetate form). The column is washed with 8 column volumes of water followed by elution of acetic acid solutions of increasing molarity. The desired mixture of 2(S),3(S)- and 2(S),3(R)-3-phenethyl aspartates are combined, concentrated, and chased with water to yield 3-phenethyl L-aspartic acid as a mixture of diastereomers.

### EXAMPLE 6

This example demonstrates the synthesis of 3-cinnamyl L-aspartic acid (Compound VIII).

N-trityl-3-cinnamyl L-aspartate dimethyl ester: The N-trityl L-aspartate dimethyl ester (1.0 eq.) dissolved in THF (SureSeal bottle) is chilled to −30° C. under argon followed by the addition of 2M lithium hexamethyldisilazide/THF solution (2.2 eq.) and is stirred at −30° C. for approximately 30 min. at which time cinnamyl bromide (2.0 eq.) is added dropwise in THF. The mixture is warmed to −5° C. and stirred for 1 hr. under argon. The reaction is then quenched with a 1M solution of ammonium chloride (2 eq.), and diluted with diethyl ether. The mixture is allowed to warm to room temperature, separated, and the organic layer washed with brine and dried with sodium sulfate. The drying agent is then filtered off using a silica plug with a 30% ethyl acetate 70% hexanes mixture used to wash the silica, and the organic solution concentrated to yield the crude product N-trityl-3-cinnamyl L-aspartate dimethyl ester containing cinnamyl bromide as a contaminant. This is used in the next step without further purification.

3-cinnamyl L-aspartic acid (VIII): To the crude N-trityl-3-cinnamyl L-aspartate dimethyl ester is added 6N HCI and the mixture is refluxed for 6 hr. The mixture is concentrated, taken up in a minimum amount of water, washed with ethyl acetate, and concentrated to remove the solvent. The residue is then taken up in a minimum amount of water, neutralized with sodium hydroxide, and loaded onto a column containing ion exchange resin (Ag-1 2×acetate form). The column is washed with 8 column volumes of water followed by elution of acetic acid solutions of increasing molarity. The desired mixture of 2(S),3(S)- and 2(S),3(R)-3-cinnamyl aspartates are combined, concentrated, and chased with water to yield 3-cinnamyl L-aspartic acid as a mixture of diastereomers.

### EXAMPLE 7

This example demonstrates the synthesis of 3-(1-methyl-4-nitronaphthalene) L-aspartic acid (Compound X).

N-trityl-3-(1-methyl4-nitronaphthalene) L-aspartate dimethyl ester: The N-trityl L-aspartate dimethyl ester (1.0 eq.) dissolved in 20 ml THF (SureSeal bottle) is chilled to −30° C. under argon followed by the addition of 2M lithium hexamethyldisilazide/THF solution (2.2 eq.) and is stirred at −30° C. for approximately 30 min., at which time 1-bromomethyl-4-nitronaphthalene (1.5 eq.) is added dropwise in THF. The mixture is warmed to −5° C. and stirred for 1 hr. under argon. The reaction is then quenched with a 1M solution of ammonium chloride (2 eq.), and diluted with diethyl ether. The mixture is allowed to warm to room temperature, separated, and the organic layer washed with brine and dried with sodium sulfate. The drying agent is then filtered off using a silica plug with a 30% ethyl acetate 70% hexanes mixture used to wash the silica, and the organic solution concentrated to yield the crude product N-trityl-3-(1-methyl-4-nitronaphthalene) L-aspartate dimethyl ester containing 1-bromomethyl-4-nitronaphthalene as a contaminant. This is then used in the next step without further purification.

3-(1-methyl-4-nitronaphthalene) L-aspartic acid (X): To the crude N-trityl-3-(1-methyl-4-nitronaphthalene) L-aspartate dimethyl ester is added 6N HCI and the mixture is refluxed for 6 hr. The mixture is concentrated, taken up in a minimum amount of water, washed with ethyl acetate, and concentrated to remove the solvent. The residue is then taken up in a minimum amount of water, neutralized with sodium hydroxide, and loaded onto a column containing ion exchange resin (Ag-1 2×acetate form). The column is washed with 8 column volumes of water followed by elution of acetic acid solutions of increasing molarity. The desired mixture of 2(S),3(S)- and 2(S),3(R)-3-(1-methyl-4-nitronaphthalene) aspartates are combined, concentrated, and chased with water to yield 3-(1-methyl-4-nitronaphthalene) L-aspartic acid as a mixture of diastereomers.

### EXAMPLE 8

This example demonstrates the pharmacological characterization of 3-benzyl L-aspartate as an inhibitor of EAAT3.

EAAT1 and EAAT3 cDNA were PCR amplified from pBlueScript-hEAAT1 and pBlueScript-hEAAT3 using primer pairs (forward; 5′ATAAGGATCCATGACTAAAAGCA-ACGGA3′ (SEQ ID NO:1) and reverse 5′TATTGATATCCTACATCTTGGTTTCACT3′ (SEQ ID NO:2)) and (forward: 5′ATAAGGATCCATGGGGAAACCGGCGAGG3′ (SEQ ID NO:3) and reverse 5′TATTGATATCCTAGAACTGTGAGGTCTG3′ (SEQ ID NO:)) respectively. Each primer pair introduced BamHI sites at the 5′ ends and EcoRV sites at the 3′ ends of each amplified fragment. The PCR fragments were then subcloned into the BamHI and EcoRV sites within the polylinker of the AAV vector pAM-CAG-WPRE to create pAM-CAG-EAAT1-WPRE and pAM-CAG-EAAT3-WPRE. Final clones were confirmed by double stranded sequencing. A 1.9 kb EcoRI fragment containing the hEAAT2 cDNA clone was subcloned from pBlueScript-hEAAT2 into the EcoRI site of pAM-CAG-WPRE by standard molecular biology techniques to create pAM-CAG-EAAT2-WPRE.

C17.2 cells between passages 10 and 20 were seeded at 1×10⁵ cells/well in 12-well plates and grown in complete Dulbecco's minimum essential medium (DMEM) supplemented with 10% fetal bovine serum, 1 mM sodium pyruvate, 0.1 mM nonessential amino acids solution, and 0.05% penicillinestreptomycin (5000 units/ml) and gentamicin sulfate (0.05 mg/ml). At 24 h after plating, cells were transfected using Lipofectamine 2000 Transfection Reagent (Invitrogen, Carlsbad, Calif.) in a ratio of 4 μl of Lipofectamine to 3 μg of purified plasmid DNA in accordance with the manufacturer's instructions. After 24 h, the relative levels of functional D-[³H]Asp uptake were determined by the method of Martin and Shain (J. Biol. Chem. 1979, 254, 7076-7084.) as described below.

Transfected C17.2 cells were grown in DMEM containing 10% fetal calf serum (FCS) in a humid atmosphere of 5% CO₂. Near-confluent cells (plated at 7×10⁴ to 1×10⁵ cells/well) were rinsed with a physiological buffer (138 mM NaCl, 11 mM D-glucose, 5.3 mM KCl, 0.4 mM KH₂PO₄, 0.3 mM Na₂HPO₄, 1.1 mM CaCl₂, 0.7 mM MgSO₄, 10 mM 4-(2-hydroxyethyl)-1-piperazineethanesulfonic acid (HEPES), pH 7.4) and allowed to preincubate at 37° C. for 5 min. Uptake was initiated by replacing the pre-incubation buffer with buffer containing D-[³H]aspartate (5-100 μM) and inhibitors (10-100 μM). Following a 5 min incubation, the media was removed by rapid suction and the cells rinsed three times with ice-cold buffer. The cells were dissolved in 0.4 N NaOH for 24 h and analyzed for radioactivity by liquid scintillation counting (LSC) and protein by the bicinchoninic acid (BCA) (Pierce) method. Transport rates were corrected for background, i.e., radiolabel accumulation at 4° C. Initial studies confirmed that uptake quantified in this manner was linear with time and protein levels and that uptake in untransfected C17.2 cells was indistinguishable from background. Kinetic analyses of the transport inhibitors were carried out using k-cat kinetic computational software (BioMetallics Inc.) to generate Lineweaver-Burk plots with curve-fit weighting based on constant relative error. Kᵢ values were also determined using this software on the basis of a replot of K_{m,app} values. The results demonstrate that 3-benzyl-aspartate is a competitive inhibitor of glutamate transport and that it exhibits preferential activity at the EAAT3 transporter subtype.

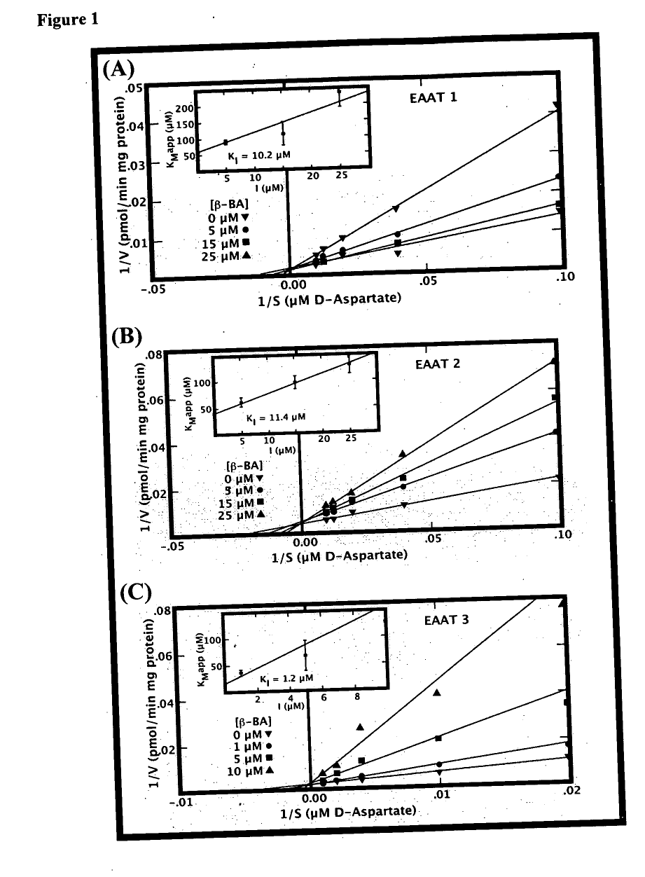

Four concentrations of 3-benzyl-aspartate (3-BA; 5 μM, 10 μM, 25 μM, 100 μM) were tested for their ability to inhibit the uptake of ³H-D-aspartate (25 μM) (Table 1). Two concentrations of 3(S)-benzyl L-aspartate (3(S)-BA; 10 μM, 100 μM) and two concentrations of 3(R)-benzyl L-aspartate (3(R)-BA; 10 μM, 100 μM) were also tested and are included in Table 1. The data are reported as % of control, thus the smaller the number, the greater the inhibition. As a positive control, the inhibitor L-β-threo-benzyl aspartate (TBOA) was also included in the study. The results show that at half the concentration of TBOA (5 vs. 10 μM), 3-benzyl-aspartate more effectively inhibited EAAT3. Representative plots from which these values were determined are shown in FIG. 1.

TABLE 1

| | EAAT1 | | | EAAT2 | | | EAAT3 | | |
|---|---|---|---|---|---|---|---|---|---|
| | mean | SD | n | mean | SD | n | mean | SD | n |
| 10 μM TBOA | 50% | 4% | 3 | 30% | 2% | 3 | 53% | 5% | 3 |
| 5 μM 3-BA | 74% | 2% | 3 | 71% | 9% | 3 | 46% | 1% | 3 |
| 10 μM 3-BA | 63% | 9% | 3 | 70% | 1% | 3 | 46% | 3% | 3 |
| 25 μM 3-BA | 50% | 9% | 3 | 39% | 5% | 3 | 20% | 4% | 3 |
| 100 μM 3-BA | 20% | 4% | 3 | 15% | 3% | 3 | 4% | 2% | 3 |
| 10 μM 3(S)-BA | 50% | 3% | 3 | 53% | 2% | 3 | 30% | 4% | 3 |
| 100 μM 3(S)-BA | 8% | 1% | 3 | 9% | 1% | 3 | 1% | 1% | 3 |
| 10 μM 3(R)-BA | 79% | 3% | 3 | 90% | 5% | 3 | 66% | 8% | 3 |
| 100 μM 3(R)-BA | 59% | 4% | 3 | 48% | 2% | 3 | 14% | 2% | 3 |

### EXAMPLE 9

This example demonstrates the antagonism of human EAAT transporter-mediated L-glutamate uptake currents in voltage-clamped Xenopus laevis oocytes microinjected with mRNA transcribed from EAAT1, EAAT2, or EAAT3 cDNA.

Capped cRNAwas transcribed from the human brain glutamate transporter EAAT1-3 cDNAs as described (Arriza, J. L.; Fairman, W. A.; Wadiche, J. I.; Murdoch, G. H.; Kavanaugh, M. P.; Amara, S. G. J. Neurosci. 1994, 14, 5559-5569.). Transcripts were microinjected into Xenopus oocytes (50 ng per oocyte) and membrane currents were recorded 3-6 days later. Ringer recording solution contained 96 mM NaCl, 2 mM KCI, 1 mM MgCI₂, 1.8 mM CaCI₂, and 5 mM HEPES (pH 7.4). Two microelectrode voltage-clamp recordings were performed at 22° C. with an Axon Instruments GeneClamp 500 amplifier interfaced to a PC using a Digidata 1200 converter controlled using the pCLAMP program suite (version 6.0; Axon Instruments). Microelectrodes were filled with 3 M KCI solution and had resistances of 0.5-2 Mohm. Steady state data were sampled at 1 kHz and low pass filtered at 500 Hz. Data were fitted by least squares using Kaleidagraph v3.0 (Synergy Software) to the Michaelis-Menten function. 3-benzyl L-aspartic acid was applied 10-30 s before glutamate for competition experiments; recovery from block was complete within 1 min following inhibitor washout.

The Kᵢ value for 3-benzyl aspartate, as well as a Kₘ value ofr ³H-D-aspartate, in each experiment. As shown below, the Kᵢ values determined for EAAT3 were consistently several-fold lower than for the other transporters, demonstrating on at EAAT3 (Tables 2-4).

TABLE 2
EAAT1

| Assay | Kᵢ (μM) | Kₘ (μM) | Vₘₐₓ (pmol/min/mg protein) |
|---|---|---|---|
| 1 | 13.1 | 42 | 260 |
| 2 | 8.5 | 16 | 179 |
| 3 | 16.2 | 30 | 566 |

TABLE 3
EAAT2

| Assay | Kᵢ (μM) | Kₘ (μM) | Vₘₐₓ (pmol/min/mg protein) |
|---|---|---|---|
| 1 | 7.8 | 20 | 603 |
| 2 | 10.6 | 23 | 683 |
| 3 | 12.9 | 36 | 2565 |

TABLE 4
EAAT3

| Assay | Kᵢ (μM) | Kₘ (μM) | Vₘₐₓ (pmol/min/mg protein) |
|---|---|---|---|
| 1 | 2.7 | 16 | 444 |
| 2 | 2.2 | 12 | 142 |
| 3 | 3.3 | 16 | 430 |
| 4 | 2.1 | 24 | 1347 |

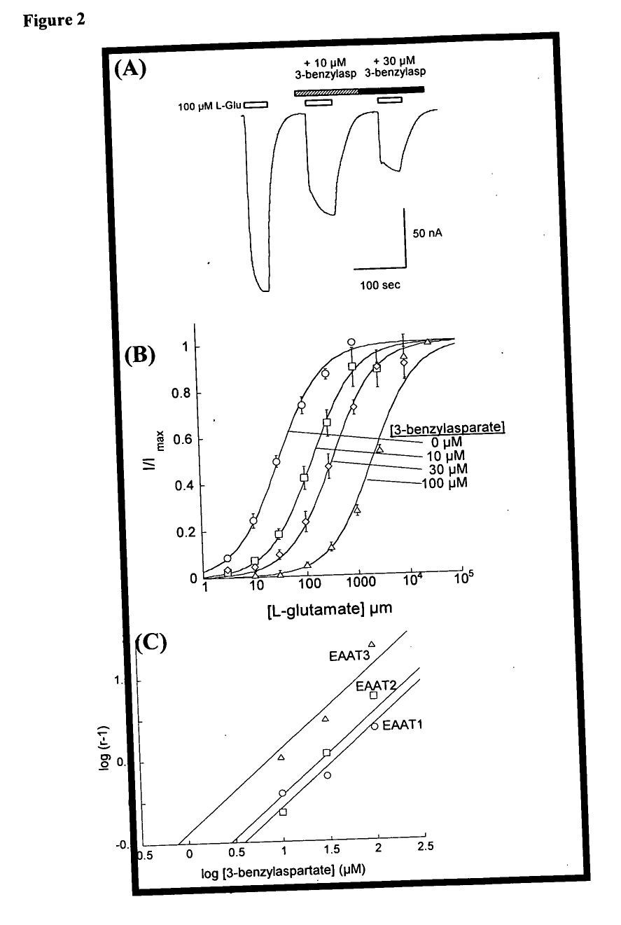

Representative plots from which these values were determined are shown in Figure 2(A-C). The patterns are consistent with a competitive mechanism of inhibition. Thus, the results indicate that not only does 3-benzyl-aspartate inhibit glutamate uptake, it does so by binding to the substrate site on the transporter.

### EXAMPLE 10

This example demonstrates the enhancement of synaptic transmission by 3-benzyl-aspartate.

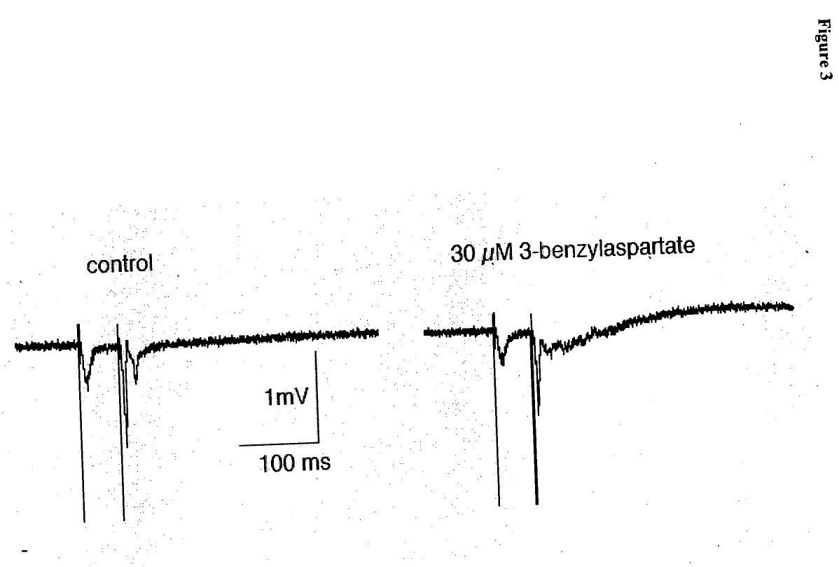

Recordings were made from 400 μm thick rat brain slices that encompass hippocampal sections. Sections were continually perfused in oxygenated artificial cerebrospinal fluid (ACSF). A stimulating electrode that delivered 100 μs pulses of variable intensity was placed in stratum radiatum, and extracellular field recordings were obtained from the CA1 dendritic field. Benzyl aspartate was applied by switching to ACSF containing 10-100 μM drug concentrations. In a paired pulse protocol, a prominent enhancement of the evoked excitatory postsynaptic potential is observed, and a prolonged NMDA receptor mediated potential is seen to be induced by benzyl aspartate. These results can be seen in FIG. 3.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCE TO MATERIAL ON COMPACT DISC

Two compact discs have been submitted with this application. The discs are identical, thus they both contain a file entitled “238772.ST25.txt”, having a size of 2.00 KB, which was created on Oct. 21, 2005. The files contained on the discs are hereby incorporated by reference.

1. An L-aspartate derivative compound represented by the following structure 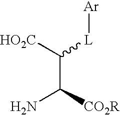
wherein Ar represents an aromatic group; L represents a linking moiety; R represents hydrogen, alkyl, aryl, or heteroaryl; and [symbol] indicates that the stereochemistry at the 3-position can be R or S.

2. The compound of claim 1 wherein L comprises an alkyl chain.

3. The compound of claim 2 wherein the alkyl chain comprises 1-3 carbon atoms.

4. The compound of claim 3 wherein the alkyl chain is a methylene.

5. The compound of claim 3 wherein the alkyl chain is an ethylene.

6. The compound of claim 1 wherein L comprises an alkenyl group.

7. The compound of claim 6 wherein the alkenyl group comprises 2-4 carbon atoms.

8. The compound of claim 7 wherein the alkenyl group is —CH₂—CH═CH—.

9. The compound of claim 1 wherein Ar comprises phenyl, naphthyl, anthracenyl, or phenanthryl.

10. The compound of claim 9 wherein the aromatic group is phenyl.

11. The compound of claim 9 wherein the aromatic group is naphthyl.

12. The compound of claim 1 wherein the aromatic group is further substituted with at least one substituent selected from the group consisting of a C₁₋₆alkyl group, a C₂₋₆alkenyl group, a C₅₋₁₀aryl group, a C₁₋₆alkoxy group, a hydroxy group, an amino group, a nitro group, a cyano group, a carboxyl group, and a halogen.

13. The compound of claim 12 wherein the aromatic group is substituted with a nitro group.

14. The compound of claim 12 wherein the aromatic group is substituted with a C₁₋₆alkyl group.

15. The compound of claim 12 wherein the aromatic group is substituted with two methyl groups.

16. The compound of claim 1 wherein the stereochemical configuration at the 3-position is R.

17. The compound of claim 1 wherein the stereochemical configuration at the 3-position is S.

18. A mixture comprising two compounds of claim 1, the first compound having the R stereochemical configuration at the 3-position and the second compound having the S stereochemical configuration at the 3-position.

19. The mixture of claim 18 wherein the R and S enantiomers exist in about a 1:1 ratio.

20. The mixture of claim 18 wherein the R and S enantiomers exist in about a 1:2 ratio.

21. The compound of claim 1 wherein Ar is phenyl, L is methylene, and R is hydrogen.

22. The compound of claim 21 wherein the stereochemical configuration at the 3-position is R.

23. The compound of claim 21 wherein the stereochemical configuration at the 3-position is S.

24. A mixture comprising two compounds of claim 21, the first compound having the R stereochemical configuration at the 3-position and the second compound having the S stereochemical configuration at the 3-position, wherein the R and S enantiomers exist in about a 1:2 ratio.

25. The compound of claim 1 wherein Ar is phenyl, L is ethylene, and R is hydrogen.

26. The compound of claim 1 wherein Ar is 3,5-dimethylphenyl, L is methylene, and R is hydrogen.

27. The compound of claim 1 wherein Ar is naphthyl, L is methylene, and R is hydrogen.

28. The compound of claim 1 wherein Ar is phenyl, L is —CH₂—CH═CH—, and R is hydrogen.

29. The compound of claim 1 wherein Ar is 4-nitrophenyl, L is methylene, and R is hydrogen.

30. The compound of claim 1 wherein Ar is 4-nitronaphthyl, L is methylene, and R is hydrogen.

31. A pharmaceutically acceptable salt, solvate, or hydrate of the compound of claim 1.

32. A method of selectively attenuating the activity of excitatory amino acid transporter 3 (EAAT3) in a cell, the method comprising providing a compound of claim 1, or a salt, solvate, or hydrate thereof, to a cell in an amount sufficient to inhibit the activity of EAAT3 within the cell.

33. A method of enhancing synaptic transmission, the method comprising administering the compound of claim 1, or a salt, solvate, or hydrate thereof, to a neural synapse in an amount sufficient to enhance transmission at the synapse.

34. A method of treating a patient suffering from a neuropathy or a neurodegenerative disease, the method comprising administering to said patient the compound of claim 1, or a salt, solvate, or hydrate thereof, in an amount sufficient to treat the neuropathy or neurodegenerative disease or symptoms thereof in said patient.

35. The method of claim 34, wherein L-glutamate transporter activity is involved in the onset of the disease.

36. A method of treating a patient suffering from Alzheimer's disease, the method comprising administering to said patient the compound of claim 1, or a salt, solvate, or hydrate thereof, in an amount sufficient to treat Alzheimer's disease or symptoms thereof in said patient.

37. A pharmaceutical composition comprising the compound of claim 1, or a salt, solvate, or hydrate thereof, and a pharmaceutically acceptable carrier.
